(19) United States
(12) Patent Application Publication
(10) Pub. No.: US 2023/0258636 A1
(43) Pub. Date: Aug. 17, 2023

(54) HYDROGEL MICROPARTICLES FOR APPLICATIONS IN CELL AND PARTICLE SEPARATION

(71) Applicant: The Charles Stark Draper Laboratory, Inc., Cambridge, MA (US)

(72) Inventors: Ryan Dubay, Cambridge, MA (US); Jason Fiering, Cambridge, MA (US); Eric Darling, Norfolk, MA (US)

(73) Assignee: The Charles Stark Draper Laboratory, Inc., Cambridge, MA (US)

(21) Appl. No.: 18/108,171

(22) Filed: Feb. 10, 2023

Related U.S. Application Data
(60) Provisional application No. 63/309,225, filed on Feb. 11, 2022.

(51) Int. Cl. G01N 33/569; B01L 3/00; G01N 1/40

(52) U.S. Cl. CPC G01N 33/56972; B01L 3/502753; G01N 1/4077; B01L 2200/0652; B01L 2300/0864; G01N 2001/4094; B01L 2400/0439

(57) ABSTRACT

A method of manufacturing synthetic particles for use in microfluidic devices is disclosed. The method includes identifying a set of particle characteristics for a fluid-based process. The set of particle characteristics can include a synthetic particle density and one or more of a size, compressibility, elastic modulus, or porosity. The method includes selecting an input material for the synthetic particles based on the set of synthetic particle characteristics. The method may include selecting an additive based on the set of synthetic particle characteristics. The method includes providing input material and the additive into a droplet generator to create one or more synthetic particles having the set of synthetic particle characteristics, and modifying a surface characteristic the synthetic particles, such that the synthetic particles bind to a target particle in a solution.

### CROSS-REFERENCES TO RELATED APPLICATIONS

This application claims the benefit of and priority to U.S. Provisional Patent Application No. 63/309,225, filed Feb. 11, 2022, the content of which is incorporated herein by reference in its entirety for all purposes.

### BACKGROUND

Devices for cell or molecule separation may utilize particles to aid their performance in various fluid-based processes. These particles may be coated or subjected to surface treatments, such that they bind to cells having a complementary surface molecule. However, such synthetic particles lack controlled characteristics in one or more of size, density, compressibility, elastic modulus, or porosity, and as a result, their behavior in fluid-based processes is unpredictable or unfavorable.

### SUMMARY

It would therefore be advantageous for techniques that allow for consistent manufacture and utilization of particles in cell or molecule separation, sorting, capture, or positioning. The present disclosure provides techniques to control the characteristics of these particles, and therefore provide predictable behavior when utilizing these particles in acoustophoresis devices or other fluid-based devices that rely on particle characteristics. The present disclosure provides improved methods for producing particles of controlled characteristics for use in applications in manipulation, separation, sorting, capture, and positioning, among others, and in particular for use in microfluidic devices that implement such processes.

At least one aspect of the present disclosure is directed to a method of manufacturing synthetic particles for use in fluid-based devices. The method includes identifying a set of synthetic particle characteristics for one or more synthetic particles. The set of synthetic particle characteristics comprise a synthetic particle density and one or more other physical properties. The method includes selecting an input material for the one or more synthetic particles based on the set of synthetic particle characteristics. The method includes providing the input material into a droplet generator to create the one or more synthetic particles having the set of synthetic particle characteristics. The method includes modifying a surface characteristic of the one or more synthetic particles to cause the one or more synthetic particles to bind to one or more target particles in a solution.

In some implementations, the one or more other physical properties include a size, compressibility, elastic modulus, or porosity for the one or more synthetic particles. In some implementations, identifying the set of synthetic particle characteristics is based on one or more attributes of the one or more target particles. In some implementations, the method includes selecting a percentage of a polymer in the input material to modulate s compressibility or the synthetic particle density of the one or more synthetic particles.

In some implementations, the one or more synthetic particles comprise a hydrogel. In some implementations, the input material comprises an additive including one or more of a lipid, a silicone, an alkane, a metal, or a metal oxide. In some implementations, the method includes providing the one or more synthetic particles with the one or more target particles in the solution, causing the one or more synthetic particles to bind with the one or more target particles.

In some implementations, identifying the set of synthetic particle characteristics is based on a desired acoustic contrast when suspended in a predetermined fluid solution. In some implementations, the set of synthetic particle characteristics comprise a first surface characteristic. In some implementations, modifying the first surface characteristic comprises applying a surface treatment including antibodies to the one or more synthetic particles.

In some implementations, the one or more target particles include one or more of T-cells or lymphocytes. In some implementations, the method includes providing the solution to a separation device. In some implementations, the solution further comprises one or more unwanted particles. In some implementations, the method includes activating the separation device to produce a first output stream comprising the one or more target particles and the one or more synthetic particles, and a second stream comprising the one or more unwanted particles.

At least one other aspect of the present disclosure is directed to a system. The system includes one or more input streams that receive input material to be provided to a droplet generator, the input material selected based on a set of synthetic particle characteristics comprising a synthetic particle density and one or more other physical properties. The system includes an output stream that receives one or more synthetic particles created by the droplet generator, the one or more synthetic particles having the set of synthetic particle characteristics. A surface characteristic of the one or more synthetic particles are modified in a reservoir that stores the one or more synthetic particles to cause the one or more synthetic particles to bind to one or more target particles in a solution.

In some implementations, the one or more other physical properties include a size, compressibility, elastic modulus, or porosity for the one or more synthetic particles. In some implementations, the input material comprises a polymer selected to modulate a compressibility or the synthetic particle density of the one or more synthetic particles. In some implementations, the one or more synthetic particles comprise a hydrogel. In some implementations, the input material comprises an additive including one or more of a lipid, a silicone, an alkane, a metal, or a metal oxide. In some implementations, the one or more synthetic particles modified according to a surface treatment including applying antibodies to the one or more synthetic particles.

Yet another aspect of the present disclosure is directed to a microfluidic device. The microfluidic device includes an input channel configured to receive a solution including one or more synthetic particles created by a droplet generator using input material. The input material is selected based on a set of synthetic particle characteristics comprising a synthetic particle density and one or more other physical properties. A surface characteristic of the one or more synthetic particles is modified to cause the one or more synthetic particles to bind to one or more target particles in the solution. The microfluidic device includes a first output channel that provides a first output stream comprising the one or more synthetic particles bound to the one or more target particles. The microfluidic device includes a second output channel that provides a second output stream comprising one or more unwanted particles in the solution.

In some implementations, the microfluidic device includes a transducer configured to actuate the central channel at a predetermined frequency.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. Aspects can be combined, and it will be readily appreciated that features described in the context of one aspect of the invention can be combined with other aspects. Aspects can be implemented in any convenient form.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. The foregoing and other objects, aspects, features, and advantages of the disclosure will become more apparent and better understood by referring to the following description taken in conjunction with the accompanying drawings, in which:

FIG. 1 illustrates a high-level block diagram showing the manufacture of synthetic particles using the techniques described herein, in accordance with one or more implementations;

FIG. 2 illustrates a flow diagram showing the use of the synthetic particles described herein in a microfluidic process, in accordance with one or more implementations; and

FIG. 3 illustrates a flow diagram of an example method of manufacturing synthetic particles for use in microfluidic devices, in accordance with one or more implementations.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure provides techniques for cell, particle, or molecule manipulation, separation, sorting, capture, or positioning using synthetic particles that are manufactured to have controlled characteristics. The use of such particles in these techniques can aid in the performance of various separation devices, such as fluid-based separation devices, by binding to target particles in a solution to change the behavior (e.g., response to acoustic actuation, etc.) of those target particles in the device. For example, synthetic particles may be coated with an affinity molecule, or another targeting agent such as an antibody or a binding protein, such that the synthetic particles bind to cells having a complementary surface molecule. Other binding techniques are also possible. In some implementations, synthetic particles may be coated or permeated with a compound intended to adsorb or “capture” a high quantity of a complementary compound in suspension, such as nucleic acid.

The synthetic particles described herein can be manufactured to have controlled characteristics, such as particle size, density, compressibility, elastic modulus, or porosity, among others. These controlled characteristics allow the particles to have predictable and desired behavior when utilized in material manipulation, separation, sorting, capturing, or positioning processes. The synthetic particles can be manufactured to have a desired motion characteristics, dispersal characteristics, or responsiveness to mechanical forces in a microfluidic device. In addition to improved methods for producing particles of controlled characteristics, the present disclosure provides techniques for the use of such controlled synthetic particles in a variety of example microfluidic devices and processes.

The synthetic particles described herein can include hydrogels or other polymer materials, and can be manufactured by means of a microfluidic “droplet generator” (e.g., a miniaturized nozzle fluidly coupled with microchannels) to provide precise control of the particle's size, compliance, or polymer fraction/porosity, among other properties. Additionally, the present disclosure provides for the addition of optional reagents, which may be used to control other particle characteristics such as density, buoyancy, speed of sound, or electrical permittivity, among others. The surface chemistry or affinity of the synthetic particles may also be modified to enhance or inhibit its binding to cells or compounds when suspended with them in solution. The synthetic particles can therefore be tailored to specific molecular or cellular applications by binding to desired particles in a solution.

The properties of the synthetic particles may be predetermined or tuned based on theoretical prediction or experimental studies to achieve desired performance in microfluidic devices, which may be used to perform processes such as manipulation, separation, sorting, capture, or positioning. The synthetic particles may be used, for example, in acoustic separation devices, where it may be desirable to use particles of a specific, known size, and acoustic impedance (or acoustic contrast) to bind to specific cell types and enhance a purification procedure. More than one synthetic particle type may be used together, where differing acoustic contrasts are employed to refine separation. For example, desired results may be achieved if one synthetic particle has positive acoustic contrast and another has negative acoustic contrast, under the anticipated operating conditions of the device. Similarly, a highly compliant particle (e.g., low elastic modulus) may be desired because of its resulting response in inertial microfluidic devices and its response to hydrodynamic velocity fields. A synthetic particle of controlled compliance could be desired for its ability to pack or displace other particles in very concentrated conditions in solution, or to promote the mechanical adhesion of a certain cell type. A particle of controlled density, size, and deformability may be desired in centrifugal manipulations to achieve a desired sedimentation rate.

The properties of the synthetic particles may also be designed or tuned to respond differently depending on conditions of their surrounding medium. For example, the synthetic particles may change the sign of their acoustic contrast upon a corresponding change of density of the medium, or upon a change in temperature of the medium. Likewise, other properties may change upon introduction of an additive to the medium, such as a change in pH of the medium caused by introducing an acidic or basic solution.

To achieve the controlled properties for synthetic particles, various manufacturing conditions can be adjusted or manipulated. Some non-limiting examples of what is adjusted in the fabrication of synthetic particles include the type of selected monomer along with specific polymerization reagents and curing conditions; the flow conditions in the droplet generator; the geometry of the nozzle or associated ducts of the droplet generator; the presence of additives such as nanoparticles of metals, metal oxides, ceramics, polymers, waxes, lipids, or the presence of post-production treatments with antibodies, adhesion molecules, or surfactants, among others.

By manufacturing and using synthetic particles with different characteristics, various molecule or cell acoustophoresis processes can be manipulated to achieve specific target particle selection. As described herein, the terms “low acoustic contrast” and “high acoustic contrast” should be understood to correspond to the relative acoustic contrast between target particles (and any synthetic particles bound thereto) and the media in which the target particles are suspended. In addition to binding the synthetic particles (which may sometimes be referred to herein as “selection particles”) to target molecules or cells, undesired (e.g., waste) particles may also be bound to specific synthetic particles to achieve desired manipulation, separation, sorting, capture, or positioning of target particles in a solution.

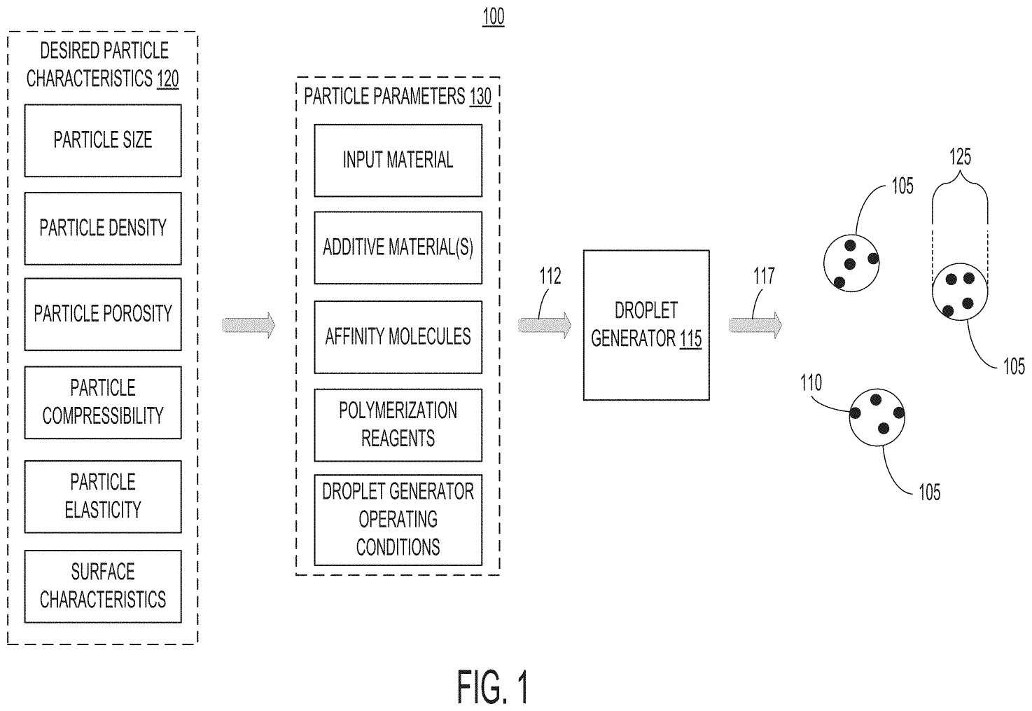

Referring now to FIG. 1, shown is an example block diagram for a process of manufacturing synthetic particles for use in microfluidic devices, in accordance with one or more implementations. As shown, the desired particle characteristics 120 are used to influence the selection of particle parameters 130 in the manufacture of the synthetic particles 105, which are created using the droplet generator 115. The desired particle characteristics 120 can include, but are not necessarily limited to, a desired particle size, a desired particle density, a desired particle compressibility (or bulk modulus), a desired particle elasticity (or modulus of elasticity), a desired particle porosity, as well as surface characteristics or treatments to allow the synthetic particles 105 to bind to desired target particles. The particle parameters 130 selected in the manufacture of the particles can include but are not necessarily limited to input material(s) (e.g., a hydrogel, monomers, polymers, etc.), additive material(s) (e.g., metal, metal oxides, lipids, polymers etc.), affinity molecules (e.g., antibodies, ligands, aptamers, etc.), polymerization reagents, along with the operating conditions of the droplet generator as is known in the art.

The synthetic particles 105 are manufactured to bind to target particles in a solution, to thereby modify the acoustic response of the target particles. As described herein, the term “binding,” in the context of binding a synthetic particle 105 to a target particle, can refer to a variety of bonding techniques. For example, synthetic particles 105 may bind to target particles using antibodies, aptamers, antigen-binding fragments, lectins, or any means of chemical bonding. Some examples of target particles can be cells (e.g., T-cells, bacteria, any cell that expresses a marker or surface feature to which a synthetic particle 105 can bind, etc.) or any molecule or compound to which a synthetic particle 105 can bind (e.g. virus, nucleic acid, protein).

The term “marker” as described herein can be any type of cellular marker or target particle marker, which may be any surface feature, molecule, protein, antibody, or other chemical or physical feature. For example, a marker can be an antibody marker or a protein present on the external cell membrane of a cell. In general, a marker can be any type of surface feature to which other particles may bind. In some implementations, cell membranes may include multiple types of cell markers, and therefore may bind to more than one type of synthetic particle 105.

Generally, the characteristics for any synthetic particles 105 may be selected based on the target particles to which the synthetic particles 105 will bind. As synthetic particles 105 are manufactured to modify the acoustic response of the target particles, the desired characteristics of the synthetic particles 105 may be chosen such that the attributes of the aggregate of bound particles is distinct from the extant attributes of the target particles alone. For example, if a target particle complex must have increased density to change its acoustic contrast to a desired value (e.g., relative to a solution), a relatively high particle density for the synthetic particles 105 that bind to the target particle can be desired. Likewise, if lowering the acoustic contrast of the target particle complex is desired, the density (and other properties that affect acoustic contrast of the synthetic particle 105) may be selected to be relatively low. Synthetic particles 105 that have a low acoustic contrast may occasionally be referred to herein as “low contrast particles,” while synthetic particles 105 that have a high acoustic contrast may occasionally be referred to as “high contrast particles.”

Other properties may also be selected to change the resulting acoustic contrast of a synthetic particle 105. For example, the compressibility of the synthetic particle 105 may be selected to provide high or low acoustic contrast. Generally, synthetic particles 105 with low compressibility have high acoustic contrast, while synthetic particles 105 with high compressibility have low acoustic contrast. The acoustic contrast of a synthetic particle 105 can be adjusted by changing the input materials used to create the synthetic particle 105. For example, the compressibility of the synthetic particle 105 can be adjusted by adjusting the percentage of a polymer in the input material used to manufacture the synthetic particles 105, or by adjusting the amount or type of additive materials in the synthetic particles 105.

The input material used to create the synthetic particles 105 can include, for example, a hydrogel polymer. Hydrogels can be cross-linked hydrophilic polymers, which may be highly absorbent. However, due to their hydrophilic nature, hydrogels retain their general structure when submerged in water. Increasing the amount of hydrogel polymers in a synthetic particle 105 relative to other materials, such as the additive materials 110, can decrease the overall compressibility of the synthetic particles 105. The percentage of hydrogel may also affect the overall density of the synthetic particle 105. Different hydrogel polymers, in connection with other materials encapsulated by the hydrogel polymers, may be selected to achieve a desired porosity for the synthetic particles 105.

To adjust the density of the synthetic particles 105, various additive materials 110 may be incorporated, such that they are encapsulated by the hydrogel polymers during the creation of the synthetic particles 105. The additive materials 110 may be nanoparticles, or other types of particles or fragments of material that are embedded in the synthetic particle 105. The additive materials 110 can occupy the remaining volume of the synthetic particles 105 (e.g., the percentage by volume) that is not occupied by hydrogel polymer(s). The additive materials 110 can include, for example, metals, metal oxides, lipids, or other types of materials. The additive materials 110 can be selected to modify various properties of the synthetic particles 105, such as the density, compressibility, or porosity of the synthetic particles 105. In some implementations, the additive materials 110 may be added to the synthetic particles 105 after polymerization. For example, the hydrogels in the synthetic particles may be temporarily swollen through the introduction of a solution, and the additive materials 110 may be introduced to and combined with the hydrogels in the synthetic particles 105 while in the temporarily swollen state.

Because many of the particle characteristics 120 affect the same properties of the synthetic particles 105, the specific values for attributes such particle size, particle density, and particle porosity, among others, are chosen to change the properties of a target particle by predetermined amounts. Some non-limiting examples of target particles can include cells, such as red blood cells or T-cells, adherent cells, or any other type of large molecule or protein to which a synthetic particle 105 can bind. In some implementations, the acoustic contrast of synthetic particles 105 or target particles may change in different solutions, which may include saline, or other density-modifying solutions such as iodixanol (Optiprep), Ficoll (polysaccharide), Histopaque, Percoll, dextran, poly(ethylene glycol), cesium chloride, and hetastarch. The synthetic particles 105 may also include one or more additive materials 110. The additive materials 110 can include gas bubbles, or oil-filled vesicles, among others. The additive materials 110 can include nanoparticles, which may be used to tune the characteristics (e.g., density, compressibility, porosity, etc.) of the synthetic particles 105. Some examples of additive materials 110 can include iron oxide, silica, cured silicone, gold, silver, or carbon, among others.

In some implementations, the additive materials 110 may reduce the effective density of the synthetic particles 105. One type of additive material 110 includes an alkane-based nanoparticle that includes a 2:1 ratio of 1-octadecane thiol to tert-butyl acrylate with 0.1% 2,2-Dimethoxy-2-phenylacetophenone (e.g., a photoinitiator). In an example manufacturing process for this type of additive material 110, the additive nanoparticles can be created via a bulk emulsion process (e.g., probe sonication) where the nanoparticles were suspended in a 1% Tween 20 solution. The additive nanoparticles can then be photopolymerized using a suitable curing light (e.g., ultra-violet light, 365 nm, etc.) during the last 5 minutes of bulk sonication followed by and additional UV exposure for 20 minutes in a curing chamber under nitrogen.

In some implementations, the additive materials 110 may include surface functionalized nanoparticles in a hydrogel or other type of input material. Such functionalized nanoparticles may include polymer or magnetic nanoparticles with antibodies. In some implementations, incorporating a surface functionalized additive material 110 may cause the synthetic particles 105 to bind to target particles in a solution without any further surface treatment of the synthetic particles 105 after they are created. In such implementations, surface modifications can performed on nanoparticle additive materials 110 prior to addition to the droplet generator 115.

The chosen particle characteristics 120 are used in connection with the droplet generator 115 to create the synthetic particles 105. The droplet generator 115 can include one or more channels, reservoirs, or other microfluidic devices or features. In some implementations, one or more input streams 112 can receive input material (e.g., the input material, the additive materials 110, etc.) to be provided droplet generator 115. The one or more input streams 112 can include one or more channels, reservoirs, or other microfluidic devices or features. The one or more input streams 112 can feed into the droplet generator 115, includes a microfluidic droplet nozzle. The synthetic particles 105 are created by feeding the input material and the additive materials 110 through the droplet generator 115, which produces a component of the synthetic particles 105 at the nozzle. The width of the nozzle of the droplet generator 115 can be modulated to achieve a desired size 125 for the synthetic particles 105. In some implementations, the synthetic particles 105 can be created using other droplet mechanisms, such as spraying, agitation, macroscale nozzles, or other emulsion processes.

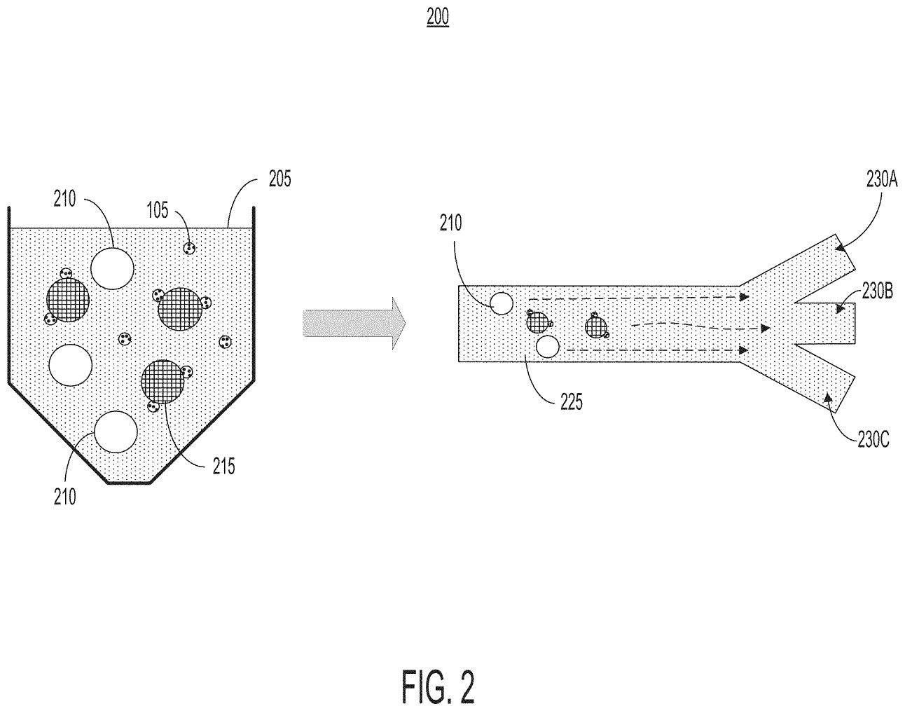

The output of the droplet generator 115 can be fed into one or more output streams 117, which can receive the synthetic particles 105 created by the droplet generator 115. The one or more output streams 117 can include one or more channels, reservoirs, or other microfluidic devices or features. In some implementations, the one or more output streams 117 may be fluidly coupled to a reservoir, channel, or other type of fluid-transporting device. For example, the output streams 117 may be fluidly coupled to a reservoir holding a solution with target particles or unwanted particles, such as the solution 205 of FIG. 2. In some implementations, the one or more output streams 117 can feed into a channel of a microfluidic device, such as the acoustophoresis device 225 of FIG. 2.

Various other characteristics of the droplet generator 115, and the input material/additives 110 provided to the droplet generator 115, can also be altered to achieve desired synthetic particle characteristics 120. For example, the types of input material or process of manufacturing the input material (e.g., a selected monomer along with specific polymerization reagents and curing conditions, etc.), the flow conditions in the droplet generator 115 (e.g., flow rate, channel size, etc.), the geometry of the nozzle or associated ducts of the droplet generator 115, as well as properties of the additive materials 110, which may include nanoparticles of metals, metal oxides, ceramics, waxes, or lipids, among others, may be controlled to achieve these characteristics. The materials exiting the nozzle can be further processed by curing steps (e.g., ultraviolet light, heat, chemical treatments), washing, filtration, or exposure to additional reagents.

Additionally, after or during their creation at the droplet generator 115, the synthetic particles 105 may be subjected to surface modifications, such that the synthetic particles 105 can bind to a desired target particle or desired target particles. Some examples of such post-production treatments include surface treatments with antibodies, adhesion molecules, or surfactants, among others. The type of post-production treatment selected for the synthetic particles 105 can be chosen based on the desired target particle to which the synthetic particle 105 will bind. In some implementations, multiple surface treatments may be applied to a synthetic particle 105, such that the synthetic particle 105 can bind to multiple target particles. Once the synthetic particles 105 have been created, they can be used on one or more separation processes, such as the microfluidic process shown in FIG. 2.

Referring now to FIG. 2, depicted is an example flow diagram 200 of a system that can be used to show the use of the synthetic particles 105 described in connection with FIG. 1 in an example microfluidic process, in accordance with one or more implementations. Although the microfluidic process shown in FIG. 2 is a continuous-flow acoustophoresis process, it should be understood that the synthetic particles 105 described herein may be utilized in any type of process that operates on target particles. In some implementations, the system of the flow diagram 200 can include any of the components described in connection with FIG. 1, including the droplet generator 115 and one or more fluid-based devices or components, including but not limited to a reservoir, pipe, a tube, a pump, among other fluid-based components or devices described herein. For example

As shown, the flow diagram 200 starts by incubating the synthetic particles 105 with target particles 215 and unwanted particles 210 in a solution 205. The microfluidic process shown in the diagram is a separation process, in which the unwanted particles 210 are separated from the target particles. However, in some implementations, the synthetic particles 105 may bind to unwanted particles rather than the target particles 215, to separate the unwanted particles 210 from the target particles using an acoustophoresis process. The solution 205 can be any type of solution, including saline, water, or another type of solution with known acoustic or physical properties. The target particles 215 can be any type of particle to which the synthetic particles 105 can bind (e.g., following a surface treatment, etc.). Some non-limiting examples of target particles 215 can include, for example, T cells, lymphocytes, or other target cells that express one or more markers to which the synthetic particles 105 can bind. In some implementations, the target particles 215 can be any type of particle or molecule to which the synthetic particles 105 can bind.

As shown, the target particles 215, the synthetic particles 105, and the unwanted particles 210, are initially suspended a solution 205. It should be understood that the particular configuration shown in the flow diagram 200 is an example implementation, and should not be considered as limiting to the types of media, particle manipulation modality, operation, or environments to which synthetic particles 105 may be bound to target cells or target particles, as described herein. In some implementations, the synthetic particles 105 may be larger than the target particles 215. In other implementations, they may be smaller. As shown, the synthetic particles 105 are incubated in the solution 205 such that the synthetic particles 105 have bound to the target particles 215. After the synthetic particles 105 have bound to the target particles 215 in the solution 205, the solution 205 can be provided as input to a separation process. The target particles 215 to which the synthetic particles 105 are bound may be referred to herein as the “bound particles 215.”

The separation process can be any type of process that is used to separate, sort, capture, or position target particles 215. As such, any type of process that leverages the altered aggregate physical properties of the target particles 215 due to the bound synthetic particles 105 may be used in connection with the synthetic particle techniques described herein. After incubating the synthetic particles 105 with the target particles 215, the solution 205 can be provided as input to a device, such as an acoustophoresis device 225. The flow diagram 200 illustrates an example acoustophoresis device 225 having an inlet, a central channel, and multiple outlet channels 230A, 230B, and 230C. The acoustophoresis device 225 can include a microfluidic channel that can receive a separation media, such as the solution 205, which can include any of the target particles 215 and the unwanted particles 210.

Using the separation techniques described herein (e.g., aided by the synthetic particles, etc.), one or more of the target particles 215 (with the synthetic particles 105 coupled thereto) can be separated from the unwanted particles 210 in the solution 205. The central channel can be a microchannel fabricated from a substrate, such as steel, silicon, glass or quartz, or a polymer with high acoustic impedance, such as polystyrene or acrylic. The central channel can be rectangular in cross section, with width and height dimensions that can range from 1 μm to 5000 μm. However, it should be understood that other sizes are possible to achieve a desired outcome. Similarly, other types of separation processes can be used in connection with the synthetic particles 105, such as centrifugal or inertial separation processes.

Furthering this example, the inlet of the acoustophoresis device 225 can receive a suspension media (e.g., the solution 205) including the target articles 215 and other, undesired particles such as the unwanted particles 210. The solution 205 can be, for example, saline, or another suitable suspension media. The solution 205 can contain additives to achieve predetermined fluid mechanical properties of the solution 205 (e.g., density, compressibility, viscosity). The inlet of the acoustophoresis device 225 can include an inlet port that may be connected or coupled to a pipe, a tube, a reservoir, a pump, or another feature that provides the solution 205), including the target particles 215 and the unwanted particles 210. In some implementations, the acoustophoresis device 225 can receive the flow of the solution 205 from another stage in a fluid-based processing pipeline, which may itself be an acoustophoresis device similar to the acoustophoresis device 225. In some implementations, the acoustophoresis device 225 may be one of many parallel acoustophoresis channels, for example, to form a system that provides increased throughput of separated cells. These parallel acoustophoresis devices 225 may operate in a multi-layer design, which provides high-throughput microfluidic separation.

The outlets 230A, 230B, and 230C of the acoustophoresis device 225 can provide streams of the solution 205 including the target particles 215 as part of the acoustophoresis separation process. In general, each of the outlets can align (e.g., along the length of the acoustophoresis channel) with a respective pressure node or pressure anti-node induced by a transducer coupled to the acoustophoresis device 225. The transducer may actuate the acoustophoresis device 225 at a predetermined frequency, which may be selected in part based on the dimensions of the acoustophoresis device 225 or the properties of the solution 205. The nodes or anti-nodes inducted in the solution 205 during the operation of the acoustophoresis device 225 can guide the target particles to a desired outlet, shown here as the outlet 230B. Likewise, the unwanted particles 210 can be guided to the other outlets 230A and 230C.

The outlets 230A, 230B, and 230C of the acoustophoresis device 225 can be outlet ports, which can be connected to (e.g., via any type of connector or fastener, etc.) a pipe, a tube, a reservoir, a pump, or another microfluidic feature that can receive an output flow of the separated suspension fluid. Although three outlets 230A, 230B, and 230C are shown, it should be understood that any number of outlets may be implemented using the techniques described herein, for example, such that each outlet aligns with a respective pressure node or anti-node induced in the suspension media by a transducer. In some implementations, any one of the outlets of the acoustophoresis device 225 can be connected (e.g., via one or more connectors, tubes, pipes, etc.) to other processing stages or other microfluidic features, as described herein. As such, the acoustophoresis device 225 may be part of a larger fluid processing system, which can include any number of microfluidic features or devices.

As described briefly above, acoustophoresis can be achieved by coupling one or more transducers (or other types of oscillators) to a wall of the microfluidic channel in the acoustophoresis device 225. The transducer can be an ultrasonic oscillator, such as a piezoelectric transducer. The transducer can be electrically driven to excite the channel of the acoustophoresis device 225 such that the target particles 215 (with the synthetic particles 105 coupled thereto) migrate toward the axial center stream of the channel as they flow through it. In this example, in the acoustophoresis device 225, the relative acoustic contrast of the target particles 215 causes the target particles 215 to migrate toward the axial center stream of the channel, and exit the acoustophoresis device 225 via the center outlet 230B. Once separated, the target particles 215 can be provided to other fluid-based devices, stored in a reservoir, or otherwise subjected to any type of post-separation process. Processes other than separation are also possible, such as trapping, concentration, buffer exchange, or washing. As shown, the relatively lower acoustic contrast of the unwanted particles 210 cause the unwanted particles 210 to migrate toward the side outlets 230A and 230C. The unwanted cells 210 can be disposed of, or similarly subjected to further processing. In some implementations, the target particles 215 may be directed toward the outer outlets 230A and 230C, while the unwanted particles are directed toward the middle outlet 230B.

A brief overview of the effect on bound target particles 215 while subjected to acoustophoresis will now be provided. The response of a particle (e.g., such as the target particles 215, or the unwanted particles 210, etc.) to manipulation by acoustophoresis is commonly predicted to behave according to the size and acoustic contrast of the particle. For example, in an example configuration in a resonant cavity the Force, F, on the particle is written as:

F∝ΦV

The relationship above provides that the force F on the particle is proportional to Φ (the acoustic contrast) and V (the particle volume). The magnitude of the force can depend on properties of the system, such as the frequency or energy applied to the acoustophoresis device 225, and the location within the resonant cavity, but these properties can be held constant to account for these differences. The acoustic contrast itself can be calculated from the particle's density and compressibility, and from the density and compressibility of the suspending fluid (e.g., the solution 205).

In the techniques described herein, the synthetic particles 105 can be used to alter the net contrast on the complex of a target particle 215 when the synthetic particle(s) 105 bind to the target particle 215. The behavior of the bound target particles 215 can be estimated based on the assumption that the effective contrast of the complex (e.g., the synthetic particle 105 bound to the target particle 215) is an average of each component's acoustic contrast weighted by its volume:

$${Φ}^{′} = \frac{{Φ}_{1} {V}_{1} + {Φ}_{2} {V}_{2} + {Φ}_{3} {V}_{3}}{{V}_{1} + {V}_{2} + {V}_{3}} ,$$

And the force on the aggregate would be estimated to vary as:

F∝Φ₁V₁+Φ₂V₂+Φ₃V₃

Where the subscripts indicate each individual particle in the aggregate. Generally, particles having a larger acoustic contrast may experience greater forces in response to actuation by a transducer during acoustophoresis. Because the acoustic contrast of a particle depends on the density and compressibility of the suspending solution 205, other configurations (e.g., synthetic particles 105 with different properties, etc.) can be utilized for different solution types. A solution 205 may be selected such that a corresponding target particle 215 (e.g., prior to binding to a synthetic particle 105) has an acoustic contrast near zero.

Similarly, adjusting the concentration of a density medium (e.g., any media having a density different from the suspension media, etc.) added to the solution (e.g., prior to, or following, any acoustophoresis processes, can be used to alter the net contrast factor of a complex of multiple target particles 215 and unwanted particles 210. In this way, multiple stages of acoustophoresis separation processes can be applied to a solution 205 to achieve optimal separation of many different types of particles. Various density mediums may be utilized with the techniques described herein. Some examples include iodixanol (Optiprep), Ficoll (polysaccharide), Histopaque, Percoll, dextran, polyethylene glycol, and cesium chloride, and hetastarch, any of which may be introduced to influence the net acoustic contrast of a target particle 215 or an unwanted particle 210.

Although the acoustophoresis device 225 shown in FIG. 2 is a continuous-flow acoustophoresis device 225, it should be understood that the techniques described herein are compatible with any type of acoustic device or device that leverages differences in characteristics (e.g., the acoustic contrast) realized through binding the synthetic particles 105 to target particle 215. For example, the techniques described herein may be implemented similarly in connection with acoustic trapping devices, surface acoustic wave devices, macroscale acoustic separators, sorting, purification, or isolation devices, or positioning devices, or combinations thereof. Because the techniques described herein operate via manipulation of the acoustic contrast of target particles 215, any device that uses acoustic contrast to manipulate particles is compatible with and may benefit from the techniques described herein.

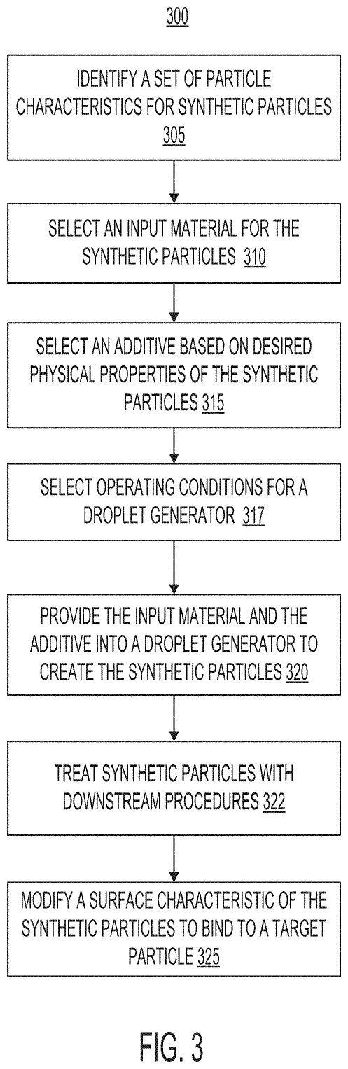

Referring to FIG. 3, depicted is a flow diagram of an example method of manufacturing synthetic particles (e.g., the synthetic particles 105 described in connection with FIGS. 1 and 2) for use in fluid-based devices (e.g., the acoustophoresis device 225 described in connection with FIG. 2), in accordance with one or more implementations. In brief overview, the method 300 can include identifying a set of particle characteristics (e.g., the desired particle characteristics 120) for synthetic particles (e.g., the synthetic particles 105, etc.) to bind to target particles (e.g., the target particles 215, etc.) for use in a manufacturing process (STEP 305), selecting an input material and quantity thereof for the synthetic particles based on desired physical properties of the synthetic particles (STEP 310), selecting an additive material (e.g., the additive material 110, etc.) based on desired synthetic particle physical properties (STEP 315), selecting operating conditions for a droplet generator (STEP 317), providing the input material and the additive materials into a droplet generator (e.g., the droplet generator 115) to create the synthetic particles (STEP 320), treating the synthetic particles with downstream procedures such as curing washing, and/or filtration (STEP 322), and modifying a surface characteristic of the synthetic particles to bind to a target particle (STEP 325).

In further detail, the method 300 includes identifying a set of particle characteristics (e.g., the particle characteristics 120) for synthetic particles (e.g., the synthetic particles 105, etc.) to bind to target particles (e.g., the target particles 215, etc.) for use in a manufacturing process (STEP 305). The set of particle characteristics can include a particle density, and one or more of a particle size, compressibility, elastic modulus, or porosity. Each of these particle characteristics can be chosen or selected based on both a target particle to which the synthetic particle will bind, and on the properties of the solution (e.g., the solution 205) in which the target particles are suspended. As described herein above, the force F on a particle suspended in a solution is proportional to Φ (the acoustic contrast) and V (the particle volume). The acoustic contrast itself can be calculated from the particle's density and compressibility, and from the density and compressibility of the suspending fluid (e.g., the solution 205). As such, the density of the synthetic particle can be chosen to modify the aggregate density of the bound target particle (e.g., total mass divided by total volume) to achieve a target acoustic contrast, which will in turn govern the behavior of the target particle in an acoustophoresis system.

The synthetic particles are used to alter the net contrast on the complex of a target particle when the synthetic particle(s) bind to the target particle. The behavior of the target particles can be estimated based on the assumption that the effective contrast of the complex (e.g., the synthetic particle bound to the target particle) is an average of each component's acoustic contrast weighted by its volume:

$${Φ}^{′} = \frac{{Φ}_{1} {V}_{1} + {Φ}_{2} {V}_{2} + {Φ}_{3} {V}_{3}}{{V}_{1} + {V}_{2} + {V}_{3}} ,$$

and the force on the aggregate would be estimated to vary as:

F∝Φ₁V₁+Φ₂V₂+Φ₃V₃

Where the subscripts indicate each individual particle in the aggregate. Generally, particles having a larger acoustic contrast may experience greater forces in response to actuation by a transducer during acoustophoresis. Because the synthetic particle characteristics can be identified in part based on one or more attributes the target particle, such as the density of the target particle, the inherent acoustic contrast of the target particle (e.g., relative to a known or predetermined solution), the mass of the target particle, the volume of the target particle, any surface chemicals, antibodies, or markers present on the target particle, or any other attributes of the target particle. Some examples of the synthetic particle characteristics can include particle size (e.g., volume, etc.), particle mass, particle density, surface treatments to apply to the particle (e.g., such that the synthetic particle will bind with a target particle), particle porosity, types of materials (e.g., input materials) that make up the synthetic particle, and any additives to encapsulate in the synthetic particle.

These synthetic particle characteristics can be selected to satisfy the aforementioned force relationships, taking into account the attributes of the target particle to which the synthetic particle will bind. The acoustic contrast of a particle can also depend on the density and compressibility of the solution in which the target particles are suspended. Some example density values for biological applications (including target particles being adipocytes or other dense cell types) can be in the range including 0.9 to greater than 2 g/mL. Some example synthetic particle sizes can range from 0.1 μm to about 100 μm or greater, or within a range of about 5 μm to about 40 μm. Some example elastic moduli for synthetic particles can range from 0.05 kPa to about 25 kPa or greater.

The method includes selecting an input material for the synthetic particles (STEP 310). Once the synthetic particle characteristics have been identified for a particular application (e.g., particular target particles, separation, isolation, or purification process, etc.), the input material for the synthetic particles can be selected. The material can be selected to achieve the identified particle characteristics, such as particle density based on particle size, volume, or other characteristics. The input materials for the synthetic particles can include, for example, a hydrogel polymer. Hydrogels are cross-linked hydrophilic polymers, which may be highly absorbent and have predetermined properties when submerged in a known solution. Increasing the amount of hydrogel polymers in a synthetic particle relative to other materials, such as nanoparticles or other additive materials, can decrease the overall compressibility of the synthetic particles. The density of the synthetic particle can be modulated by adjusting the amount of the hydrogel in the synthetic particle (e.g., to achieve a predetermined mass for a predetermined volume percentage of the synthetic particle, etc.). Similarly, the types of hydrogels, and the curing conditions used to create the hydrogels, can be modulated to achieve the identified synthetic particle characteristics. In some implementations, multiple input materials may be used (e.g., monomers, polymers, hydrogels, etc.) in varying percentages to achieve the identified synthetic particle characteristics, such as density, compressibility, elastic modulus, size, or porosity. Selecting the input material can include selecting a percentage of a polymer (e.g., a hydrogel, etc.) in the input material to modulate the compressibility or density of the synthetic particles.

The method includes selecting an additive material (e.g., the additive material 110, etc.) based on a desired synthetic particle density (STEP 315). After selecting the input material, additive materials can be selected to modulate the density, compressibility, electric transmissivity, or other characteristics of the synthetic particles. The additive materials may be nanoparticles or other types of particles or fragments of material that are embedded in the synthetic particle. The additive materials can occupy the remaining volume of the synthetic particles (e.g., the percentage by volume) that is not occupied by hydrogel polymer(s). The additive materials can include, for example, metals, metal oxides, lipids, or other types of materials. The additive materials can be selected to modify various properties of the synthetic particles, such as the density, compressibility, or porosity of the synthetic particles.

The percentage and type of additives can be selected to achieve the identified synthetic particle characteristics, such as a desired particle density at a predetermined particle size. Adding denser additive materials, such as metals or metal oxides, increases the density of the synthetic particles, while adding less dense additive materials, such as lipids or oils, generally decreases the density of the synthetic particles. In some implementations, the synthetic particles may not include any additive materials, and may instead be constructed solely from polymers, such as hydrogels. The additive materials can be encapsulated by the input material during or after the manufacture of the synthetic particles. The additive materials can be incorporated in the manufacture either before or after selecting the operating conditions for the droplet generator.

The method includes selecting operating conditions for a droplet generator (STEP 317). The operating conditions for the droplet generator may affect various properties of the synthetic particles created using the droplet generator, such as synthetic particle size. Some example operating conditions of the droplet generator can include the flow conditions in the droplet generator (e.g., flow rate, etc.), channel size, or the geometry of the nozzle or associated ducts of the droplet generator. These operating conditions may be chosen to achieve the desired synthetic particle characteristics identified in prior steps.

The method includes providing the input material and the additive materials into a droplet generator (e.g., the droplet generator 115) to create the synthetic particles (STEP 320). The droplet generator can include one or more channels, reservoirs, and other features, which feed into a droplet nozzle. The synthetic particles are created by feeding the input material and the additive materials through the droplet generator, which produces the synthetic particles at the nozzle. The input materials may include a solution, or other features or chemicals used to cure the polymers in the input material. The width of the nozzle of the droplet generator can be modulated to achieve a desired size for the synthetic particles, in accordance with the predetermined particle density or volume. In some implementations, the synthetic particles can be created using other droplet mechanisms, such as spraying, agitation, macroscale nozzles, or other emulsion processes.

The method may include treating the synthetic particles with downstream procedures (STEP 322). After the droplet generator produces the synthetic particles, the particles may be subjected to additional procedures to achieve the desired synthetic particle characteristics. Some example downstream procedures can include curing washing, or filtration, among others. Curing the synthetic particles can include exposing the synthetic particles to ultraviolet light, heat, or chemical treatments. Additionally, the synthetic particles may be subjected to a filtration to isolate synthetic particles having desired particle characteristics from synthetic particles that may not have the desired particles characteristics. The synthetic particles may also be exposed to additional reagents, which may change the characteristics of the materials that make up the synthetic particles after the synthetic particles are produced by the droplet generator.

Various other characteristics of the droplet generator, and the input material or additives provided as input to the droplet generator, are controlled achieve the identified synthetic particle characteristics. For example, the flow conditions in the droplet generator (e.g., flow rate, channel size, etc.), the geometry of the nozzle or associated ducts of the droplet generator, as well as properties of the additive materials, which may include nanoparticles of metals, metal oxides, ceramics, waxes, or lipids, among others, may be controlled to achieve these characteristics. In some implementations, if an application requires multiple types of synthetic particles (e.g., for a solution containing multiple target particles, or for target particles that will bind to more than one synthetic particle, etc.), STEPS 305-322 of the method 300 can be repeated for each synthetic particle type. In such implementations, the particle characteristics of each synthetic particle may depend in part on the characteristics of other synthetic particles (e.g., in the case that multiple synthetic particles bind to a single target particle). After production, the synthetic particles may be introduced into a solution containing one or more target particles, or may be subjected to surface treatments in STEP 325.

The method includes modifying a surface characteristic of the synthetic particles to bind to a target particle (STEP 325). After their creation, the synthetic particles may be subjected to post-production treatments with antibodies, adhesion molecules, surfactants, or other types of surface treatments to allow the synthetic particles to bind to one or more target particles. Generally, target particles that are cells can express one or more surface markers or antibodies. The synthetic particles can be subjected to one or more post-production treatments with corresponding antibodies. These surface treatments allow the synthetic particles to bind to the corresponding markers on the target cells, thereby attaching the synthetic particle to the target particle and altering its aggregate physical properties. Similar approaches can be performed to bind the synthetic particles to non-organic particles or molecules. In some implementations, multiple surface treatments can be applied to a synthetic particle such that it may bind to multiple target particles in a solution.

Once the synthetic particles have been created and treated, the synthetic particles can be introduced into the same solution as the target particles to which they will bind. If the target particles are cells, the synthetic particles may bind with a particular protein on the target cell during incubation. The solution including the synthetic particles and the target particles may be subjected to external forces, such as heat, movement, or other forces, to encourage binding of the synthetic particles to the target particles. In the case where the target molecules are not cells or organisms, the synthetic particles may bind to an affinity molecule present on the surface of the target particles. In general, the synthetic particles may bind to target particles that are cells using any of antibodies, aptamers, antigen-binding fragments, lectins, or other biochemistry-related molecules that allow a synthetic particle to bind to a surface or portion of a target particle. In some implementations, the target cells and the synthetic particles can be suspended in a solution that facilitates binding of the synthetic particles to the target particles.

After binding the synthetic particles to target particles in a solution, the solution can be provided as input to a microfluidic device, such as the acoustophoresis device 225 described in connection with FIG. 2. Although the present disclosure has described the use of synthetic particles to aid in particle manipulation, separation, sorting, capture, or positioning, it should be understood that any type of process that leverages differences in particle characteristics may benefit from the present techniques. Providing the bound particles as input to a fluid-based process may include introducing a density media to the solution including the target particles bound to the synthetic particles. Doing so can alter the density and compressibility of the solution, and thereby alter the relative force experienced by each target particle when exposed to acoustophoresis or other forces. In implementations, the process may be a multi-stage process. One such example is an acoustophoresis device, which can separate particles according to their acoustic contrast. By providing the target particles, which are treated with and bound to synthetic particles, to an acoustophoresis system, precise and high-throughput cell separation can be achieved.

In addition to the examples relating to acoustic manipulation described herein, synthetic particles can be used in other flow-based mechanisms for cell manipulation, for example in inertial separation. Inertial effects on fluid suspensions of cells and particles may be significant in fluidic systems, and these effects can be used to manipulate particles and to accomplish cell separation or concentration. In inertial fluidic systems, the channel dimensions and features may be fixed according to predetermined designs and the suspension is flowed through them at an optimum flow rate to obtain desired results.

One example of how inertial separation is exploited is as follows. Rigid particles may experience two competing “inertial” or hydrodynamic forces: wall-induced forces which act to displace a particle away from channel walls, and shear-induced forces, which displace particles along the gradient in shear rate, often toward channel walls. Cells or particles therefore can reach an equilibrium position within the channel (e.g., a focusing node) where the two competing forces are balanced. On the other hand, more elastic particles may deform in flow, and as a result their equilibrium position may occur in a different location within the channel when compared with rigid particles. For example, in some cases less rigid particles are displaced to position further from channel walls than more rigid particles. Furthermore, as in acoustic separation, inertial separation is sensitive to particle size.

Synthetic particles with controlled properties, such as those described herein, can be conjugated with target cells to alter the cells' behavior in an inertial fluidic device, since a cell-particle complex will have a different effective size and effective rigidity (e.g., deformability) than an unbound cell. The particle's size, elasticity, and density can be designed according to the principles of inertial separation, using the techniques described herein.

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the terms “about” and “substantially” will be understood by persons of ordinary skill in the art and will vary to some extent depending upon the context in which they are used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation or embodiment, and references to “an implementation,” “some implementations,” “one implementation,” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description, or any claim are followed by reference signs, the reference signs have been included to increase the intelligibility of the drawings, detailed description, and claims. Accordingly, neither the reference signs nor their absence has any limiting effect on the scope of any claim elements.

The devices, systems, and methods described herein may be embodied in other specific forms without departing from the characteristics thereof. The foregoing implementations are illustrative rather than limiting of the described devices, systems, and methods. Scope of the devices, systems, and methods described herein is thus indicated by the appended claims, rather than the foregoing description, and changes that come within the meaning and range of equivalency of the claims are embraced therein.

What is claimed is:

1. A method of manufacturing synthetic particles for use in fluid-based devices, comprising:
identifying a set of synthetic particle characteristics for one or more synthetic particles, wherein the set of synthetic particle characteristics comprise a synthetic particle density and one or more other physical properties;
selecting an input material for the one or more synthetic particles based on the set of synthetic particle characteristics;
providing the input material into a droplet generator to create the one or more synthetic particles having the set of synthetic particle characteristics; and
modifying a surface characteristic of the one or more synthetic particles to cause the one or more synthetic particles to bind to one or more target particles in a solution.

2. The method of claim 1, wherein the one or more other physical properties include a size, compressibility, elastic modulus, or porosity for the one or more synthetic particles.

3. The method of claim 1, wherein identifying the set of synthetic particle characteristics is based on one or more attributes of the one or more target particles.

4. The method of claim 1, further comprising selecting a percentage of a polymer in the input material to modulate s compressibility or the synthetic particle density of the one or more synthetic particles.

5. The method of claim 1, wherein the one or more synthetic particles comprise a hydrogel.

6. The method of claim 1, wherein the input material comprises an additive including one or more of a lipid, a silicone, an alkane, a metal, or a metal oxide.

7. The method of claim 1, further comprising providing the one or more synthetic particles with the one or more target particles in the solution, causing the one or more synthetic particles to bind with the one or more target particles.

8. The method of claim 1, wherein identifying the set of synthetic particle characteristics is based on a desired acoustic contrast when suspended in a predetermined fluid solution.

9. The method of claim 1, wherein the set of synthetic particle characteristics comprise a first surface characteristic, and wherein modifying the first surface characteristic comprises applying a surface treatment including antibodies to the one or more synthetic particles.

10. The method of claim 1, wherein the one or more target particles include one or more of T-cells or lymphocytes.

11. The method of claim 1, further comprising providing the solution to a separation device.

12. The method of claim 11, wherein the solution further comprises one or more unwanted particles, and the method further comprises activating the separation device to produce a first output stream comprising the one or more target particles and the one or more synthetic particles, and a second stream comprising the one or more unwanted particles.

13. A system, comprising:
one or more input streams that receive input material to be provided to a droplet generator, the input material selected based on a set of synthetic particle characteristics comprising a synthetic particle density and one or more other physical properties;
an output stream that receives one or more synthetic particles created by the droplet generator, the one or more synthetic particles having the set of synthetic particle characteristics; and
wherein a surface characteristic of the one or more synthetic particles are modified in a reservoir that stores the one or more synthetic particles to cause the one or more synthetic particles to bind to one or more target particles in a solution.

14. The system of claim 13, wherein the one or more other physical properties include a size, compressibility, elastic modulus, or porosity for the one or more synthetic particles.

15. The system of claim 13, wherein the input material comprises a polymer selected to modulate a compressibility or the synthetic particle density of the one or more synthetic particles.

16. The system of claim 13, wherein the one or more synthetic particles comprise a hydrogel.

17. The system of claim 13, wherein the input material comprises an additive including one or more of a lipid, a silicone, an alkane, a metal, or a metal oxide.

18. The system of claim 13, wherein the one or more synthetic particles modified according to a surface treatment including applying antibodies to the one or more synthetic particles.

19. A microfluidic device, comprising:
an input channel configured to receive a solution including one or more synthetic particles created by a droplet generator using input material, the input material selected based on a set of synthetic particle characteristics comprising a synthetic particle density and one or more other physical properties, wherein a surface characteristic of the one or more synthetic particles is modified to cause the one or more synthetic particles to bind to one or more target particles in the solution;
a first output channel that provides a first output stream comprising the one or more synthetic particles bound to the one or more target particles; and
a second output channel that provides a second output stream comprising one or more unwanted particles in the solution.

20. The microfluidic device of claim 19, further comprising a transducer configured to actuate the central channel at a predetermined frequency.
